(19) United States
(12) Patent Application Publication
(10) Pub. No.: US 2012/0213866 A1
(43) Pub. Date: Aug. 23, 2012

(54) METHOD AND SYSTEM FOR MODULATING SUBSTANCES AFFECTING BODILY PROCESSES

(76) Inventor: WILLIAM J. McFAUL, JACKSON, NJ (US)

(21) Appl. No.: 13/403,221

(22) Filed: Feb. 23, 2012

Related U.S. Application Data
(60) Provisional application No. 61/445,716, filed on Feb. 23, 2011.

(51) Int. Cl. A61K 33/26; A61P 43/00; A61K 35/00

(52) U.S. Cl. 424/646; 514/789

(57) ABSTRACT

A method for establishing a treatment for promoting the formation of substances in an individual. The measured or assessed level of substances in the body are compared in order to determine the treatment to provide to an individual.

This application claims the benefit of U.S. Provisional Application Ser. No. 61/445,716 filed Feb. 23, 2011, the contents of which are incorporated herein by reference.

### BACKGROUND OF THE INVENTION

1. Field of the Invention

The present invention relates to the field of testing for the corollary relationships between bodily substances and their interactions. In particular, the present invention is directed to the method and system for signaling molecules in the body.

2. Description of the Related Technology

All forms of life are supported by a variety of substances; including but not limited to hormones, proteins, peptides, amino acids, elements (minerals), protein kinases, vitamins (chemicals or compounds that function as signaling molecules) and bacteria. These substances create the homeostasis essential to maintain life for humans as well as plants and animals. It should be noted that many hormones and proteins are signaling molecules.

Associated substances affect levels of each other in the body. Additionally, certain other substances cooperate with the levels of the associated substances. Furthermore, certain substances are a part of more than one group that affects the substances. These relationships can be utilized in order to determine various health parameters of an individual, however, to date; these corollary relationships have not been established in order to effectively use them.

Therefore, there is a need in the field to identify and assess the homeostatic relationship between the substances that are important to the body's functioning via the measuring of various levels of the substances that constitute the associated substances. In lay terms, if a bodily substance (e.g. a signaling molecule) were comprised of eggs, flour and milk; the levels of each would determine the outcome (efficacy). Numerous factors can influence the outcomes; examples include size of the eggs, altitude, freshness and type of the milk, etc. If the quantities of each constituent are not within a particular range, outcomes will be impacted. These same scenarios apply to the body's signaling molecules. Current methods do not exist for the assessment of current levels of each individual's existing micronutrients that will establish the need for replenishment of essential substances such as particular amino acids or elements (e.g. those listed on the Period Chart). There is also a need to utilize the homeostatic relationships in order to develop methods and systems for promoting the formation of personalized therapeutic strategies to ensure wellness; i.e. a healthy individual.

### SUMMARY OF THE INVENTION

Another object of the invention is to identify the specific substances that must be in balance (homeostasis) in promoting the formation of substances; e.g. signaling molecules.

Another object of the invention is a method of establishing tests to determine imbalances between the relationships of substances that can affect bodily processes.

Another object of the invention is to determine the interactions between substances such as those between proteins and the outcomes when signaling molecules are not adequately formed to facilitate their interactions.

A method of for providing homeostasis of SIRT 3 via the levels of substances that form SIRT 3 in an individual comprising: measuring a level of iron in the individual; measuring a level of sulfur in the individual; measuring a level of phosphorus in the individual; comparing the measured levels; and establishing homeostasis of SIRT 3 based upon the measured levels.

A system for providing homeostasis of SIRT 3 via the levels of substances that form SIRT 3 in an individual comprising means for measuring a level of iron in the individual comprising; means for measuring a level of phosphorous in the individual; means for measuring a level of iron in the individual; means for comparing the measured levels; and means for establishing homeostasis of SIRT 3 based upon the measured levels. Should this say a system to provide the results of . . .

Still yet another aspect of the present invention may be a method for establishing a test to assess the existing levels of the constituents necessary to form the signaling molecule SIRT 1 in an individual comprising: measuring a level of phosphorus in the individual; measuring a level of lysine in the individual; measuring a level of sulfur in the individual; comparing the measured levels and means for establishing a treatment based upon the measured levels.

Still yet another aspect of the present invention may be a method for establishing a test to assess the existing levels of the constituents necessary to form the signaling molecule SIRT 2 in an individual comprising: measuring a level of phosphorus in the individual; measuring a level of lysine in the individual; measuring a level of silicon in the individual; comparing the measured levels and means for establishing a treatment based upon the measured levels.

Still yet another aspect of the present invention may be a method for establishing a test to assess the existing levels of the constituents necessary to form the signaling molecule SIRT 4 in an individual comprising: measuring a level of phosphorus in the individual; measuring a level of cysteine in the individual; measuring a level of sulfur in the individual; comparing the measured levels and means for establishing a treatment based upon the measured levels.

Still yet another aspect of the present invention may be a method for establishing a test to assess the existing levels of the constituents necessary to form the signaling molecule SIRT 5 in an individual comprising: measuring a level of phosphorus in the individual; measuring a level of cysteine in the individual; measuring a level of silicon in the individual; comparing the measured levels and means for establishing a treatment based upon the measured levels.

Still yet another aspect of the present invention may be a method for establishing a test to assess the existing levels of the constituents necessary to form the signaling molecule SIRT 6 in an individual comprising: measuring a level of phosphorous in the individual; measuring a level of cysteine in the individual; measuring a level of magnesium in the individual; comparing the measured levels; and establishing a treatment based upon the measured levels.

Still yet another aspect of the present invention may be a method for establishing a establishing a test to assess the existing levels of the constituents necessary to form the signaling molecule SIRT 7 in an individual comprising: measuring a level of phosphorous in the individual; measuring a level of lysine in the individual; measuring a level of magnesium in the individual; comparing the measured levels; and establishing a treatment based upon the measured levels.

Still yet another aspect of the present invention may be a method for establishing a test to assess the existing levels of the constituents that form one of the signaling molecules for cellular respiration in an individual comprising: measuring a level of sulfur in the individual; measuring a level of nickel in the individual; measuring a level of iron in the individual; comparing the measured levels; and establishing a treatment based upon the measured levels.

Still yet another aspect of the present invention may be a method for establishing a Still yet another aspect of the present invention may be a method for establishing a test to assess the existing levels of the constituents that form another signaling molecule for cellular respiration in an individual comprising: measuring a level of sulfur in the individual; measuring a level of molybdenum in the individual; measuring a level of iron in the individual; comparing the measured levels; and establishing a treatment based upon the measured levels.

Still yet another aspect of the present invention may be a method for establishing a test to assess the existing levels of the constituents that form a signaling molecule for blood oxidation in an individual comprising: measuring a level of sulfur in the individual; measuring a level of histidine in the individual; measuring a level of iron in the individual; comparing the measured levels; and establishing a treatment based upon the measured levels.

Still yet another aspect of the present invention may be a method for establishing a test to assess the existing levels of the constituents that form a signaling molecule for iron metabolism in blood of an individual comprising: measuring a level of copper in the individual; measuring a level of zinc in the individual; measuring a level of iron in the individual; comparing the measured levels; and establishing a treatment based upon the measured levels.

Still yet another aspect of the present invention may be a method for establishing a test to assess the existing levels of the constituents that form the signaling molecule eumelanin comprising: measuring a level of copper in the individual; measuring a level of serine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule phenomelanin comprising: measuring a level of copper in the individual; measuring a level of threonine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule neuromelanin comprising: measuring a level of copper in the individual; measuring a level of cysteine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule that is one of the forms of vitamin B6 comprising: measuring a level of copper in the individual; measuring a level of aspartic acid in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule that is another one of the forms of vitamin B6 comprising: measuring a level of copper in the individual; measuring a level of alanine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule that is another one of the forms of vitamin B6 comprising: measuring a level of copper in the individual; measuring a level of glutamic acid in the individual; measuring a level of zinc in the individual; comparing the measured levels; and establishing a treatment based upon the measured levels.

Still yet another aspect of the present invention may be a method for establishing a test to assess the existing levels of cyanocobalamin in an individual comprising: measuring a level of cobalt in the individual; measuring a level of alanine in the individual; measuring a level of cyanide in the individual; comparing the measured levels; and establishing a treatment based upon the measured levels.

Still yet another aspect of the present invention may be a method for establishing a test to assess the existing levels of proteins for cell adhesion in an individual comprising: measuring a level of DSC3 in the individual; measuring a level of DSC2 in the individual; measuring a level of DSG1 in the individual; comparing the measured levels; and establishing a treatment based upon the measured levels.

Still yet another aspect of the present invention may be a method for establishing a test to assess the existing levels of proteins for cell adhesion in an individual comprising: measuring a level of DSC1 in the individual; measuring a level of DSC2 in the individual; measuring a level of DSC4 in the individual; comparing the measured levels; and establishing a treatment based upon the measured levels.

Still yet another aspect of the present invention may be a method for establishing a establishing a test to assess the existing levels of the constituents that form the signaling molecule for cadmium chelation in an individual comprising: measuring a level of zinc in the individual; measuring a level of cysteine in the individual; measuring a level of maganese in the individual; comparing the measured levels; and establishing a treatment based upon the measured levels.

Still yet another aspect of the present invention may be a method for establishing a establishing a test to assess the existing levels of the constituents that form the signaling molecule for connective tissue formation in an individual comprising: measuring a level of silicon in the individual; measuring a level of calcium in the individual; measuring a level of gold in the individual; comparing the measured levels; and establishing a treatment based upon the measured levels.

Still yet another aspect of the present invention may be a method for establishing a test to assess the existing levels of the constituents that form the signaling molecule for connective tissue formation in an individual comprising: measuring a level of silicon in the individual; measuring a level of quartz in the individual; measuring a level of oxygen in the individual; comparing the measured levels; and establishing a treatment based upon the measured levels.

Still yet another aspect of the present invention may be a method for establishing a test to assess the existing levels of the constituents that form the signaling molecule dopamine in an individual: measuring a level of nickel in the individual comprising; measuring a level of phenylalanine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule norepinephrine comprising; measuring a level of nickel; measuring a level of tyrosine; measuring a level of zinc; and establishing a treatment based upon the measured levels.

Still yet another aspect of the present invention may be a method for establishing a test to assess the existing levels of the constituents that form the signaling molecule adrenaline comprising; measuring a level of nickel; measuring a level of tryptophan; measuring a level of zinc; and establishing a treatment based upon the measured levels.

Still yet another aspect of the present invention may be a method for establishing a test to assess the existing levels of signaling molecules for connective tissue in an individual comprising: measuring a level of thrombospondin 1 in the individual; measuring a level of thrombospondin 2 in the individual; measuring a level of thrombospondin 3 in the individual; comparing the measured levels; and establishing a treatment based upon the measured levels.

Still yet another aspect of the present invention may be a method for establishing a test to assess the existing levels of signaling molecules for connective tissue in an individual comprising: measuring a level of thrombospondin 4 in the individual; measuring a level of thrombospondin 2 in the individual; measuring a level of thrombospondin 5 in the individual; comparing the measured levels; and establishing a treatment based upon the measured levels.

Still yet another aspect of the present invention may be a method for establishing a test to assess the existing levels of proteins for synapse formation in an individual comprising: measuring a level of NLGN3 in the individual; measuring a level of NLGN2 in the individual; measuring a level of NLGN5 in the individual; comparing the measured levels; and establishing a treatment based upon the measured levels.

Still yet another aspect of the present invention may be a method for establishing a test to assess the existing levels of proteins for synapse formation in an individual comprising: measuring a level of NLGN1 in the individual; measuring a level of NLGN2 in the individual; measuring a level of NLGN4X in the individual; comparing the measured levels; and establishing a treatment based upon the measured levels.

Still yet another aspect of the present invention may be a method for establishing a test to assess the existing levels of signaling molecules for synapse adhesion in an individual comprising: measuring a level of NRXN2 in the individual; measuring a level of NRXN1 in the individual; measuring a level of NGF in the individual; comparing the measured levels; and establishing a treatment based upon the measured levels.

Still yet another aspect of the present invention may be a method for establishing a test to assess the existing levels of signaling molecules for synapse adhesion in an individual comprising: measuring a level of NRXN3 in the individual; measuring a level of NRXN1 in the individual; measuring a level of NRXN4 in the individual; comparing the measured levels; and establishing a treatment based upon the measured levels.

Still yet another aspect of the present invention may be a method for establishing a test to assess the existing levels of the constituents that form the signaling molecule vitamin K1 in an individual comprising: measuring a level of phosphorous in the individual; measuring a level of isoleucine in the individual; measuring a level of calcium in the individual; comparing the measured levels; and establishing a treatment based upon the measured levels.

Still yet another aspect of the present invention may be a method for establishing a test to assess the existing levels of the constituents that form the signaling molecule vitamin K2 in an individual comprising: measuring a level of phosphorous in the individual; measuring a level of leucine in the individual; measuring a level of calcium in the individual; comparing the measured levels; and establishing a treatment based upon the measured levels.

Still yet another aspect of the present invention may be a method for establishing a test to assess the existing levels of the constituents that form the signaling molecule MK4 form of vitamin K in an individual comprising: measuring a level of phosphorous in the individual; measuring a level of valine in the individual; measuring a level of calcium in the individual; comparing the measured levels; and establishing a treatment based upon the measured levels.

Still yet another aspect of the present invention may be a method for establishing a test to assess the existing levels of the constituents that form one of the forms of the signaling molecule vitamin B1 in an individual comprising: measuring a level of sulfur in the individual; measuring a level of phenylalanine in the individual; measuring a level of iron in the individual; comparing the measured levels; and establishing a treatment based upon the measured levels.

Still yet another aspect of the present invention may be a method for establishing a test to assess the existing levels of the constituents that form another of the forms of the signaling molecule vitamin B1 in an individual comprising: measuring a level of sulfur in the individual; measuring a level of tyrosine in the individual; measuring a level of iron in the individual; comparing the measured levels; and establishing a treatment based upon the measured levels.

Still yet another aspect of the present invention may be a method for establishing a test to assess the existing levels of the constituents that form another of the forms of the signaling molecule vitamin B1 in an individual comprising: measuring a level of sulfur in the individual; measuring a level of tryptophan in the individual; measuring a level of iron in the individual; comparing the measured levels; and establishing a treatment based upon the measured levels.

Still yet another aspect of the present invention may be a method for establishing a test to assess the existing levels of the constituents that form one of the forms of the signaling molecule vitamin B2 in an individual comprising: measuring a level of sulfur in the individual; measuring a level of leucine in the individual; measuring a level of iron in the individual; comparing the measured levels; and establishing a treatment based upon the measured levels.

Still yet another aspect of the present invention may be a method for establishing a test to assess the existing levels of the constituents that form another of the forms of the signaling molecule vitamin B2 in an individual comprising: measuring a level of sulfur in the individual; measuring a level of isoleucine in the individual; measuring a level of iron in the individual; comparing the measured levels; and establishing a treatment based upon the measured levels.

Still yet another aspect of the present invention may be a method for establishing a test to assess the existing levels of the constituents that form another one of the forms of the signaling molecule vitamin B2 in an individual comprising: measuring a level of sulfur in the individual; measuring a level of valine in the individual; measuring a level of iron in the individual; comparing the measured levels; and establishing a treatment based upon the measured levels.

Still yet another aspect of the present invention may be a method for establishing a test to assess the existing levels of the constituents that form one of the forms of the signaling molecule vitamin B3 in an individual comprising: measuring a level of iron in the individual; measuring a level of phenylalanine in the individual; measuring a level of manganese in the individual; comparing the measured levels; and establishing a treatment based upon the measured levels.

Still yet another aspect of the present invention may be a method for establishing a test to assess the existing levels of the constituents that form another of the forms of the signaling molecule vitamin B3 in an individual comprising: measuring a level of iron in the individual; measuring a level of tyrosine in the individual; measuring a level of manganese in the individual; comparing the measured levels; and establishing a treatment based upon the measured levels.

Still yet another aspect of the present invention may be a method for establishing a test to assess the existing levels of the constituents that form another one of the forms of the signaling molecule vitamin B3 in an individual comprising: measuring a level of iron in the individual; measuring a level of tryptophan in the individual; measuring a level of manganese in the individual; comparing the measured levels; and establishing a treatment based upon the measured levels.

Still yet another aspect of the present invention may be a method for establishing a test to assess the existing levels of the constituents that form one of the forms of the signaling molecule vitamin B5 in an individual comprising: measuring a level of copper in the individual; measuring a level of histidine in the individual; measuring a level of zinc in the individual; comparing the measured levels; and establishing a treatment based upon the measured levels.

Still yet another aspect of the present invention may be a method for establishing a test to assess the existing levels of the constituents that form another of the forms of the signaling molecule vitamin B5 in an individual comprising: measuring a level of copper in the individual; measuring a level of arginine in the individual; measuring a level of zinc in the individual; comparing the measured levels; and establishing a treatment based upon the measured levels.

Still yet another aspect of the present invention may be a method for establishing a test to assess the existing levels of the constituents that form another of the forms of the signaling molecule vitamin B5 in an individual comprising: measuring a level of copper in the individual; measuring a level of lysine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule vitamin B12 in an individual comprising: measuring a level of manganese in the individual; measuring a level of cyanocobalamin in the individual; measuring a level of iron in the individual; comparing the measured levels; and establishing a treatment based upon the measured levels.

Still yet another aspect of the present invention may be a method for establishing a test to assess the existing levels of the constituents that form the signaling molecule melatonin in an individual comprising: measuring a level of tryptophan in the individual; measuring a level of nitric oxide in the individual; measuring a level of phosphorus in the individual; comparing the measured levels; and establishing a treatment based upon the measured levels.

Still yet another aspect of the present invention may be a method for establishing a test to assess the existing levels of the constituents that form the signaling molecule choline in an individual comprising; measuring a level of phosphorous in the individual; measuring a level of lysine in the individual; measuring a level of calcium in the individual; comparing the measured levels; and establishing a treatment based upon the measured levels.

Still yet another aspect of the present invention may be a method for establishing a test to assess the existing levels of the constituents that form one of the forms of the signaling molecule glutathione peroxidase in an individual comprising: measuring a level of selenium in the individual; measuring a level of threonine in the individual; measuring a level of zinc in the individual; comparing the measured levels; and establishing a treatment based upon the measured levels.

Still yet another aspect of the present invention may be a method for establishing a test to assess the existing levels of the constituents that form another one of the forms of the signaling molecule glutathione peroxidase in an individual comprising: measuring a level of selenium in the individual; measuring a level of serine in the individual; measuring a level of zinc in the individual; comparing the measured levels; and establishing a treatment based upon the measured levels.

Still yet another aspect of the present invention may be a method for establishing a test to assess the existing levels of the constituents that form another one of the forms of the signaling molecule glutathione peroxidase in an individual comprising: measuring a level of selenium in the individual; measuring a level of cysteine in the individual; measuring a level of zinc in the individual; comparing the measured levels; and establishing a treatment based upon the measured levels.

Still yet another aspect of the present invention may be a method for establishing a test to assess the existing levels of the constituents that form one of the forms of the signaling molecule vitamin B7 in an individual comprising: measuring a level of copper in the individual; measuring a level of leucine in the individual; measuring a level of zinc in the individual; comparing the measured levels; and establishing a treatment based upon the measured levels.

Still yet another aspect of the present invention may be a method for establishing a test to assess the existing levels of the constituents that form another one of the forms of the signaling molecule vitamin B7 in an individual comprising: measuring a level of copper in the individual; measuring a level of isoleucine in the individual; measuring a level of zinc in the individual; comparing the measured levels; and establishing a treatment based upon the measured levels.

Still yet another aspect of the present invention may be a method for establishing a test to assess the existing levels of the constituents that form another one of the forms of the signaling molecule vitamin B7 in an individual comprising: measuring a level of copper in the individual; measuring a level of valine in the individual; measuring a level of zinc in the individual; comparing the measured levels; and establishing a treatment based upon the measured levels.

Still yet another aspect of the present invention may be a method for establishing a test to assess the existing levels of proteins for cell proliferation and adhesion in an individual comprising: measuring a level of an EGFR2; measuring a level of an EGFR1; measuring a level of GRB7; and establishing a treatment based upon the measured levels.

Still yet another aspect of the present invention may be a method for establishing a test to assess the existing levels of proteins for cell proliferation in an individual comprising: measuring a level of EGFR3; measuring a level of EGFR1; measuring a level of EGFR4; and establishing a treatment based upon the measured levels.

Still yet another aspect of the present invention may be a method for establishing a test to assess the existing levels of the constituents that form the signaling molecule Estradiol (E2) comprising; measuring a level of selenium; measuring a level of phenylalanine; measuring a level of zinc; and establishing a treatment based upon the measured levels.

Still yet another aspect of the present invention may be a method for establishing a test to assess the existing levels of the constituents that form the signaling molecule Estrone (E1) comprising; measuring a level of selenium; measuring a level of tyrosine; measuring a level of zinc; and establishing a treatment based upon the measured levels.

Still yet another aspect of the present invention may be a method for establishing a test to assess the existing levels of the constituents that form the signaling molecule Estriol (E3) comprising; measuring a level of selenium; measuring a level of tryptophan; measuring a level of zinc; and establishing a treatment based upon the measured levels.

Still yet another aspect of the present invention may be a method for establishing a test to assess the existing levels of the constituents that form the signaling molecule serotonin comprising; measuring a level of sulfur; measuring a level of histidine; measuring a level of iron; and establishing a treatment based upon the measured levels.

Still yet another aspect of the present invention may be a method for establishing a test to assess the existing levels of the constituents that form the signaling molecule oxytocin comprising; measuring a level of sulfur; measuring a level of arginine; measuring a level of iron; and establishing a treatment based upon the measured levels.

Still yet another aspect of the present invention may be a method for establishing a test to assess the existing levels of the constituents that form the signaling molecule prolactin comprising; measuring a level of sulfur; measuring a level of lysine; measuring a level of iron; and establishing a treatment based upon the measured levels.

Still yet another aspect of the present invention may be a method for establishing a test to assess the existing levels of the constituents that form the signaling molecule cortisol comprising; measuring a level of zinc; measuring a level of nitric oxide; measuring a level of nickel; and establishing a treatment based upon the measured levels.

Still yet another aspect of the present invention may be a method for establishing a test to assess the existing levels of the constituents that form the Ergosterol form of signaling molecule vitamin D comprising: measuring a level of phosphorus in the individual; measuring a level of threonine in the individual; measuring a level of calcium in the individual; comparing the measured levels; and establishing a treatment based upon the measured levels.

Still yet another aspect of the present invention may be a method for establishing a test to assess the existing levels of the constituents that form signaling molecule vitamin D2 comprising: measuring a level of phosphorus in the individual; measuring a level of serine in the individual; measuring a level of calcium in the individual; comparing the measured levels; and establishing a treatment based upon the measured levels.

Still yet another aspect of the present invention may be a method for establishing a test to assess the existing levels of the constituents that form signaling molecule vitamin D3 comprising: measuring a level of phosphorus in the individual; measuring a level of cysteine in the individual; measuring a level of calcium in the individual; comparing the measured levels; and establishing a treatment based upon the measured levels.

Still yet another aspect of the present invention may be a method for establishing a test to assess the existing levels of the constituents that form one of the forms of signaling molecule vitamin B9 comprising: measuring a level of phosphorus in the individual; measuring a level of serine in the individual; measuring a level of sulfur in the individual; comparing the measured levels; and establishing a treatment based upon the measured levels.

Still yet another aspect of the present invention may be a method for establishing a test to assess the existing levels of the constituents that form another one of the forms of signaling molecule vitamin B9 comprising: measuring a level of phosphorus in the individual; measuring a level of threonine in the individual; measuring a level of sulfur in the individual; comparing the measured levels; and establishing a treatment based upon the measured levels.

Still yet another aspect of the present invention may be a method for establishing a test to assess the existing levels of the constituents that form the signaling molecule Fas comprising: measuring a level of calcium in the individual; measuring a level of histidine in the individual; measuring a level of magnesium in the individual; comparing the measured levels; and establishing a treatment based upon the measured levels.

Still yet another aspect of the present invention may be a method for establishing a test to assess the existing levels of the constituents that form the signaling molecule Ras comprising: measuring a level of calcium in the individual; measuring a level of arginine in the individual; measuring a level of magnesium in the individual; comparing the measured levels; and establishing a treatment based upon the measured levels.

Still yet another aspect of the present invention may be a method for establishing a test to assess the existing levels of the constituents that form the signaling molecule Bax comprising: measuring a level of calcium in the individual; measuring a level of lysine in the individual; measuring a level of magnesium in the individual; comparing the measured levels; and establishing a treatment based upon the measured levels.

Still yet another aspect of the present invention may be a method for establishing a test to assess the existing levels of the constituents that form the signaling molecule Ax1 comprising: measuring a level of nickel in the individual; measuring a level of phenylalanine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule Tyro3 comprising: measuring a level of nickel in the individual; measuring a level of tryosine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule Mer comprising: measuring a level of nickel in the individual; measuring a level of tryptophan in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule Histone H3.1 comprising: measuring a level of nickel in the individual; measuring a level of histidine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule Histone H3.2 comprising: measuring a level of nickel in the individual; measuring a level of arginine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule Histone H3.3 comprising: measuring a level of nickel in the individual; measuring a level of lysine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule Pregnenolone (PREG) comprising: measuring a level of calcium in the individual; measuring a level of glutamic acid in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule Dehydroepiandrosterone (DHEA) comprising: measuring a level of calcium in the individual; measuring a level of alanine in the individual; measuring a level of zinc in the individual; comparing the measured levels; and establishing a treatment based upon the measured levels.

Still yet another aspect of the present invention may be a method for establishing a test to assess the existing levels of the constituents that form the signaling molecule Progesterone (PROG) comprising: measuring a level of calcium in the individual; measuring a level of aspartic acid in the individual; measuring a level of zinc in the individual; comparing the measured levels; and establishing a treatment based upon the measured levels.

Still yet another aspect of the present invention may be a method for establishing a test to assess the existing levels of proteins for the formation of factors in cell proliferation and differentiation comprising: measuring a level of transforming growth factor b-2 (TGFb-2); measuring a level of transforming growth factor b-3 (TGFb-3); measuring a level of p53; and establishing a treatment based upon the measured levels.

Still yet another aspect of the present invention may be a method for establishing a test to assess the existing levels of the constituents that form the signaling molecule Interleukin 4 comprising: measuring a level of nickel; measuring a level of zinc; measuring a level of boron; and establishing a treatment based upon the measured levels.

Still yet another aspect of the present invention may be a method for establishing a test to assess the existing levels of the constituents that form the signaling molecule Interleukin 6 comprising: measuring a level of calcium; measuring a level of magnesium; measuring a level of chloride; and establishing a treatment based upon the measured levels.

Still yet another aspect of the present invention may be a method for establishing a test to assess the existing levels of the constituents that form the signaling molecule Interleukin 10 comprising: measuring a level of bromine; measuring a level of iodine; measuring a level of chloride; and establishing a treatment based upon the measured levels.

Still yet another aspect of the present invention may be a method for establishing a test to assess the existing levels of proteins for the formation of factors in cell proliferation and differentiation comprising: measuring a level of transforming growth factor b-4 (TGFb-4); measuring a level of transforming growth factor b-3 (TGFb-3); measuring a level of transforming growth factor b-1 (TGFb-1); and establishing a treatment based upon the measured levels.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

### BRIEF DESCRIPTION OF THE DRAWINGS

FIG. 1 shows the relationship between sulfur, phosphorous and iron.

FIG. 2 is a flow chart of the method for establishing a treatment for restoring homeostasis based upon the relationship of the substances shown in FIG. 1.

FIG. 3 is a schematic diagram showing the system for measuring the levels of substances from within an individual.

FIG. 4 shows the method of using the system for measuring the levels of substances within an individual, in accordance with an embodiment of the present invention.

### DETAILED DESCRIPTION OF THE PREFERRED EMBODIMENT(S)

Associated substances create the homeostasis essential to maintain life for humans as well as animals. These substances can modulate each other in various ways.

Several terms are used herein and are defined as follows:

The term “modulator” means an agent(s), substance(s) or mechanism(s) that facilitates the maintenance of homeostasis between associated substances. “Associated substances” may be hormones, elements listed on the Periodic Chart, proteins, amino acids, bacteria, virus, gases and natural processes. When associated substances are “modulated” they can decrease intensity of stimulatory processes or substances, or increase intensity of compensatory ones. When a substance is indicated as being a “modulator” it means that it plays a role along with the other associated substances in establishing a homeostatic balance. It should be understood that while one substance may be indicated as the modulator, it is the interaction between all three substances that affects the homeostatic levels.

The term “catalyst” may mean the driver, which initiates modulation to take place. Such catalysts can include signaling molecules, hormones, protein kinases, gases and countless other signaling mechanisms.

The term “Homeostatic Relationship (Homeostasis)” means the balance or equilibrium between substances. The relationship does not have to be 50%-50% but can vary depending upon the substances in question.

The term “Extracellular Matter (ECM)” means all body fluid outside of cells and blood; including but not limited to cerebrospinal fluid, lymph, chyle, interstitial fluid, saliva, tears, transcellular fluid, amniotic fluid, sperm, and sputum. These substances will also include breath as a source for testing.

The term “Mental Functioning Activity (MFA)” means the rate of activity attributable to the neural firing rate in the brain and elsewhere in the body.

The invention addresses the diagnostic analysis of the corollary relationships between substances that provide and maintain homeostasis in an individual and affect bodily processes.

The data for measuring homeostasis between substances with associated substances can vary from one individual or specimen to another due to environmental factors, basic metabolism, genetic make-up and other factors. Data derived from the application of the invention will allow for diagnostic analysis and control of levels of the substances in order to prevent their depletion, to maintain adequate levels to prevent disruption as well as to create recommendations for which substances may counteract with others and the levels of which substances must be modified on an ongoing basis in order to provide optimal health/well being through homeostasis.

Since signaling molecules are essential to create and maintain defenses for the organs, glands and operating systems of the body, as well as to provide the energy to sustain life, establishing corollary relationships between the substances necessary for their production is critical. Tests for the corollary relationship between the substances that form the signaling molecules will enable analysis to prevent depletion of the body's defense in order to prevent illness as well as to increase levels of nutritional energy to strengthen the body to offset the effects of existing ailments and diseases.

Some examples of associated substances that operate within an individual are; iron and sulfur in conjunction with phosphorus having the role/process in/for the formation of SIRT3 (sirtuin3); phosphorus and lysine in conjunction with sulfur having the role/process in/for the formation of SIRT1 (sirtuin1); Phosphorus and lysine in conjunction with silicon having the role/process in/for the formation of SIRT2 (sirtuin2); Phosphorus and cysteine in conjunction with sulfur having the role/process in/for the formation of SIRT4 (sirtuin4); Phosphorus and cysteine in conjunction with silicon having the role/process in/for the formation of SIRT5 (sirtuin5); Phosphorus and cysteine in conjunction with magnesium having the role/process in/for the formation of SIRT6 (sirtuin6); Phosphorus and lysine in conjunction with magnesium having the role/process in/for the formation of SIRT7 (sirtuin7); Sulfur and nickel in conjunction with iron having the role/process in/for aerobic cellular respiration; Sulfur and molybdenum in conjunction with iron having the role/process in/for aerobic cellular respiration; Sulfur and histidine in conjunction with iron having the role/process in/for blood oxygenation; Copper and zinc in conjunction with iron having the role/process in/for iron metabolism in blood

Additional examples are Copper and serine in conjunction with zinc having the role/process in/for formation of eumelanin; Copper and threonine in conjunction with zinc having the role/process in/for formation of phenomelanin; Copper and cysteine in conjunction with zinc having the role/process in/for formation of neuromelanin; Copper and aspartic aid in conjunction with zinc having the role/process in/for the formation of one of the forms of vitamin B6; Copper and alanine in conjunction with zinc having the role/process in/for the formation of another form of vitamin B6; Copper and glutamic acid in conjunction with zinc having the role/process in/for the formation of another form of vitamin B6; Cobalt and alanine in conjunction with cyanide having the role/process in/for the formation of cyanocobalamin; the proteins DSC3 and DSC2 in conjunction with the protein DSG1 having the role/process in/for cell adhesion; the proteins DSC1 and DSC2 the in conjunction with protein DSC4 having the role/process in/for cell adhesion

Additional examples are Zinc and cysteine in conjunction with manganese having the role/process in/for cadmium chelation; Silicon and calcium in conjunction with gold having the role/process in/for connective tissue formation; Silicon and quartz in conjunction with oxygen having the role/process in/for connective tissue formation; Nickel and phenylalanine in conjunction with zinc having the role/process in/for the formation of dopamine; Nickel and tyrosine in conjunction with zinc having the role/process in/for the formation of norepinephrine; Nickel and tryptophan in conjunction with zinc having the role/process in/for the formation of adrenaline; Thrombospondin 1 and thrombospondin 2 in conjunction with thrombospondin 3 having the role/process in/for connective tissue signaling; Thrombospondin 4 and thrombospondin 2 in conjunction with thrombospondin 5 having the role/process in/for connective tissue signaling; proteins NLGN3 and NLGN2 in conjunction with NLGN5 having the role/process in/for synapse formation; protein NLGN1 and NLGN2 in conjunction with NLGN4X having the role/process in/for synapse formation; NRXN2 and NRXN1 in conjunction with NGF having the role/process in/for adhesion of synapses; NRXN3 and NRXN1 in conjunction with NRXN4 having the role/process in/for adhesion of synapses.

Additional examples are Phosphorus and tyrosine in conjunction with calcium having the role/process in/for formation of vitamin K1; Phosphorus and leucine in conjunction with calcium having the role/process in/for the formation of vitamin K2; Phosphorus and valine in conjunction with calcium having the role/process in/for the formation of the MK4 form of vitamin K; Sulfur and phenylalanine in conjunction with iron having the role/process in/for the formation of one of the forms of vitamin B1; Sulfur and tyrosine in conjunction with iron having the role/process in/for the formation of another form of vitamin B1; Phosphorus and tryptophan in conjunction with iron having the role/process in/for the formation of another form of vitamin B1; Sulfur and leucine in conjunction with iron having the role/process in/for the formation of one of the forms of vitamin B2; Sulfur and isoleucine in conjunction with iron having the role/process in/for the formation of another form of vitamin B2; Sulfur and valine in conjunction with iron having the role/process in/for the formation of another form of vitamin B2.

Additional examples are Iron and phenylalanine in conjunction with manganese having the role/process in/for the formation of one of the forms of vitamin B3; Iron and tyrosine in conjunction with manganese having the role/process in/for the formation of another form of vitamin B3; Iron and tryptophan in conjunction with manganese having the role/process in/for the formation of another form of vitamin B3; Copper and histidine in conjunction with zinc having the role/process in/for the formation of one of the forms of vitamin B5; Copper and arginine in conjunction with zinc having the role/process in/for the formation of another form of vitamin B5; Copper and lysine in conjunction with zinc having the role/process in/for the formation of another form of vitamin B5; Manganese and cyanocobalamin in conjunction with iron having the role/process in/for the formation of vitamin B12; Tryptophan and nitric oxide in conjunction with phosphorus having the role/process in/for the formation of melatonin; Phosphorus and lysine in conjunction with calcium having the role/process in/for the formation of choline; Selenium and threonine in conjunction with zinc having the role/process in/for the formation of one of the forms of glutathione peroxidase; Selenium and serine in conjunction with zinc having the role/process in/for the formation of another form of glutathione peroxidase; Selenium and cysteine in conjunction with zinc having the role/process in/for the formation of another form of glutathione peroxidase.

Additional examples are Copper and leucine in conjunction with zinc having the role/process in/for the formation of one of the forms of vitamin B7, Copper and isoleucine zinc in conjunction with having the role/process in/for the formation of another form of vitamin B7; Copper and valine in conjunction with zinc having the role/process in/for the formation of another form of vitamin B7; Protein EGFR2 and EGFR1 in conjunction with GRB7 having the role/process in/for cell proliferation; Proteins EGFR3 and EGFR1 in conjunction with EGFR 4 having the role/process in/for cell proliferation; Selenium and phenylalanine in conjunction with zinc having the role/process in/for the formation of Estradiol (E2); Selenium and tyrosine in conjunction with zinc having the role/process in/for the formation of Estrone (E1); Selenium and tryptophan in conjunction with zinc having the role/process in/for the formation of Estriol (E3).

Additional examples are Sulfur and histidine in conjunction with iron having the role/process in/for the formation of selenium; Sulfur and arginine in conjunction with iron having the role/process in/for the formation of oxytocin; Sulfur and lysine in conjunction with iron having the role/process in/for the formation of prolactin; Zinc and nitric oxide in conjunction with iron having the role/process in/for the formation of cortisol; Phosphorus and threonine in conjunction with calcium for the formation of the Ergosterol form of vitamin D; Phosphorus and serine in conjunction with calcium having the role/process in/for the formation of vitamin D2; Phosphorus and cysteine in conjunction with calcium having the role/process in/for the formation of vitamin D3; Phosphorus and serine in conjunction with sulfur having the role/process in/for the formation of one of the forms of vitamin B9; Phosphorus and threonine in conjunction with sulfur having the role/process in/for the formation of another form of vitamin B9.

Additional examples are Calcium and histidine in conjunction with magnesium having the role/process in/for the formation of Fas; Calcium and arginine in conjunction with magnesium having the role/process in/for the formation of Ras; Calcium and lysine in conjunction with magnesium having the role/process in/for the formation of Bax; Nickel and phenylalanine in conjunction with zinc having the role/process in/for the formation of Ax1; Nickel and tyrosine in conjunction with zinc having the role/process in/for the formation of Tyro3; Nickel and tryptophan in conjunction with zinc having the role/process in/for the formation of Mer; Nickel and histidine zinc in conjunction with having the role/process in/for the formation of Histone H3.1; Nickel and arginine in conjunction with zinc having the role/process in/for the formation of Histone H3.2; Nickel and lysine in conjunction with zinc having the role/process in/for the formation of Histone H3.3.

Additional examples are Calcium and glutamic acid in conjunction with zinc having the role/process in/for the formation of Pregnenolone (PREG); Calcium and alanine in conjunction with zinc having the role/process in/for the formation of Dehydroepiandrosterone (DHEA); Calcium and aspartic acid in conjunction with zinc having the role/process in/for the formation of Progesterone (PROG); Proteins transforming growth factor beta2 and transforming growth factor beta3 in conjunction with protein p53 having the role/process in/for cellular density; transforming growth factor beta4 and transforming growth factor beta3 in conjunction with transforming growth factor betal having the role/process in/for cellular density; Nickel and zinc in conjunction with boron having the role/process in/for the formation of Interleukin 4; Calcium and magnesium in conjunction with chloride having the role/process in/for the formation of Interleukin 6; Bromine and iodine in conjunction with chloride having the role/process in/for the formation of Interleukin 10; Proteins transforming growth factor b-4 and transforming growth factor b-3 in conjunction with transforming growth factor b-1 having the role/process in/for cellular density.

It should be noted that some substances can function as part of a group of associated substances and also be a modulator for another group of associated substances. Additionally, it should be understood that while specific processes and roles are noted above as being affected by the associated substances, other processes and roles may also be affected. It should also be noted that while an example is discussed below, it is exemplary of the process that may be obtained with all listed associated substances.

Correlation between base values expressed when the range of homeostasis is determined and individual test results can be used as a means of assessing levels or disruptions between associated substances or the existence of increases in the levels of substances in order to maintain homeostasis. Methods of diagnostic analysis may also detect the presence of catalysts as markers for the existence of a disruption within a associated substance that the body is in the process of correcting. Detection of such catalysts may be included as a factor in the process of analyzing the correlation of substances within associated substances.

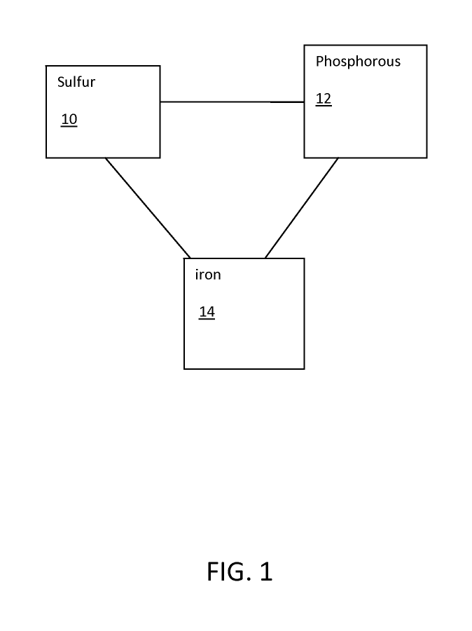

Described herein is an example of the determination of homeostatic levels of the sets of associated substances discussed above. The first set is the pair iron 10 and sulfur 12, which forms a homeostatic relationship with phosphorus 14. This relationship is shown in FIG. 1. This relationship plays a role in the formation of sirtuin 3 (SIRT 3) and the homeostatic establishment of it.

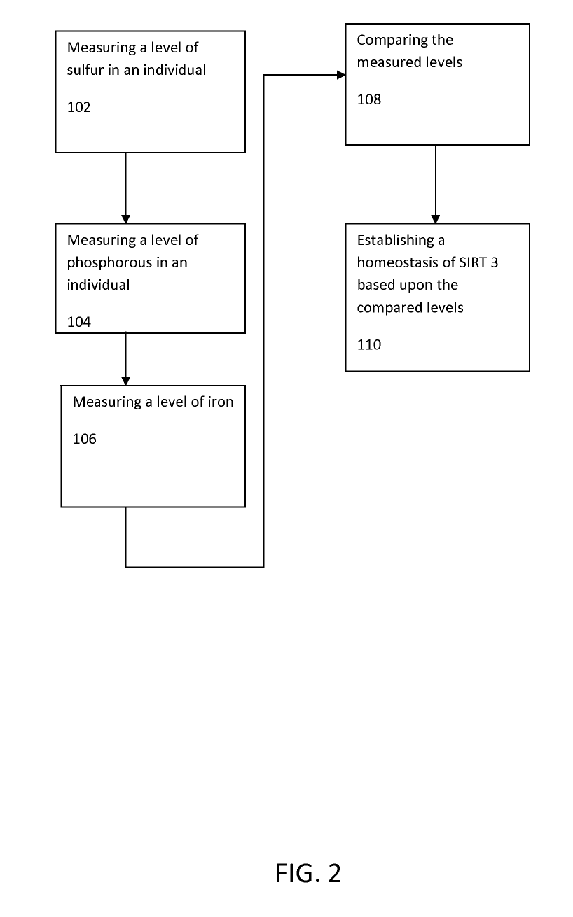

FIG. 2 shows the method of evaluating the homeostatic levels of iron 10 and sulfur 12 in order to provide a base index for administering medication and performing analysis of an individual. In step 102, the level of iron 10 is measured in an individual. The level of sulfur 12 may be measured by a variety of diagnostic tests that include, but are not limited to the analysis of diagnostic imaging that indicates activity and the analysis of various fluids and excretions within and produced by the body. Examples of these fluids and excretions include: blood and its components; serum; urine; fecal matter; collagen; chyle; interstitial fluid (tissue fluid); lymph; extracellular fluid; amniotic fluid; sweat tears; saliva; mucus; phlegm; hair; fingernails; bone marrow and breath.

In addition to the conventional diagnostic analysis of levels of substances from the aforementioned sources, various existing and yet to be developed diagnostic processes that measure outcomes resulting from these substances can be utilized. These devices may include EEG, PET scans, use of MEG machines, SPECT analysis, functional and diffusion MRI technologies as well as other iterations thereof (fMRI and dMRI respectively), CT scans, ultrasound and breath analysis devices.

Correlation between base values expressed when the range of homeostasis is determined and individual test results may be used as a means of assessing levels or disruptions between associated substances or the existence of increases in the levels of modulators in order to maintain homeostasis. Methods of diagnostic analysis may also detect the presence of catalysts as markers for the existence of a disruption within a modulated associated substance that the body is in the process of remediating. Detection of such catalysts is included as a factor in the process of analyzing the correlation of substances within modulated associated substances.

This invention provides the biological foundation that will enable monitoring relationships within modulated associated substances of substances as they relate to medical care; including wellness, prevention and treatment pertaining to the mind (brain) and body.

The measured level of iron 10 in the body or environment may be expressed in various units depending upon the test employed in order to measure the level of the sulfur 12. This level can be expressed by the variable X. There is a preferred range in which X may fall that varies depending upon the type of test that is used in order to measure the level of iron 10. In an embodiment of the present invention a standard type of test is used, such as a blood test.

In step 104, the level of phosphorous 14 in an individual is measured. This level can be measured using one of the various processes listed above. The measured level of phosphorous 14 is preferably expressed in the same units of measurement as that used in the measurement of iron 10 so as to more readily compare the levels with respect to each other in order to obtain a base level. This level can be expressed by the variable Y. There is a preferred range in which Y may fall that varies depending upon the type of test that is used in order to measure the level of phosphorous 14. In an embodiment of the present invention a standard type of test is used, such as a blood test.

In step 106, the level of iron 10 in an individual is measured. This level can be measured using one of the various processes listed above. The measured level of iron 10 is preferably expressed in the same units of measurement as that used in the measurement of sulfur 12 so as to more readily compare the levels with respect to each other in order to obtain a base level. This level can be expressed by the variable Z. There is a preferred range in which Z may fall that varies depending upon the type of test that is used in order to measure the level of iron 10. In an embodiment of the present invention a standard type of test is used, such as blood test.

In step 108, the measured levels X, Y and Z are then compared to standard levels. This may be done in a number of different ways. One possible way in which this can be accomplished is to total the values of all of the levels (X+Y+Z) in order to establish sum level C. It is also possible to establish acceptable ratios for the values X:Y, X:Z and Y:Z. The sum level C can be used in step 110 in order to establish a treatment or diagnosis of the individual that can be used in determining the provision of medication and other matters related to the measured levels. In particular the levels may be used to determine if more iron 14 may need to be supplied in order to place the level of sulfur 12 or phosphorous 14 back to proper levels and to promote the formation of SIRT 3 and establish homeostasis.

An example of this is provided below using hypothetical numbers so as to make understanding of the process easier.

In the process a level of sulfur 12 is determined from analyzing a blood test. Measuring sulfur 12 via a blood test results in a value for X of 6 in the appropriate units. Acceptable ranges for X may be between 5-10. Measuring phosphorous 14 via a blood test results in a number for Y of 5, in the appropriate units, from within a range of 1-10. Measuring iron 14 via the blood test results in a number for Z of 15, in the appropriate units, from within an acceptable range of between 1-10. These numbers are totaled and result in a C value of 26. A value for C between 10 and 20 may indicate a normal level.

A value for C that falls below 10 or above 20 may signal the need to provide a treatment that administers additional iron 10, or phosphorous 14 or iron 10 to the individual. Alternatively, it may signal that the diet should be altered in order to reduce the levels of these substances. In this instance it levels of iron 10 may be reduced. Alternatively, if the level for Z was 5, and the levels for X and Y remained the same, pills containing iron 10 may be administered in order to increase levels.

Monitoring of the individual may be performed periodically until the levels return to accepted ranges. Various means of administration are contemplated herein in addition to pill form, such as intravenous, etc. Alternatively, one of the substances may be removed from an individual's diet. It is also contemplated that one of the other substances, such as iron 10 or phosphorous 14 may be added or removed depending upon their respective levels during the initial analysis.

The assessment of the modulated substances enables the provision of consulting/counseling services relative to the biological foundation for and between the substances associated with the affected substance, in the instance of iron 10, sulfur 12 and phosphorus 14, that substance is SIRT 3.

The relationships are studied using devices that will measure levels of the substances contained within the body that are listed above. The devices may be used to assess the corollary relationships between the substances in order to compare them to standard biodiagnostic baseline assessment.

The usage of these methods may also be used for assessment of homeostatic imbalances of relationships within modulated associated substances that can enable therapies to correct these disruptions instead of waiting to the point where mental or physical consequences will require expensive or sophisticated treatment of ailments or diseases that are the outcomes.

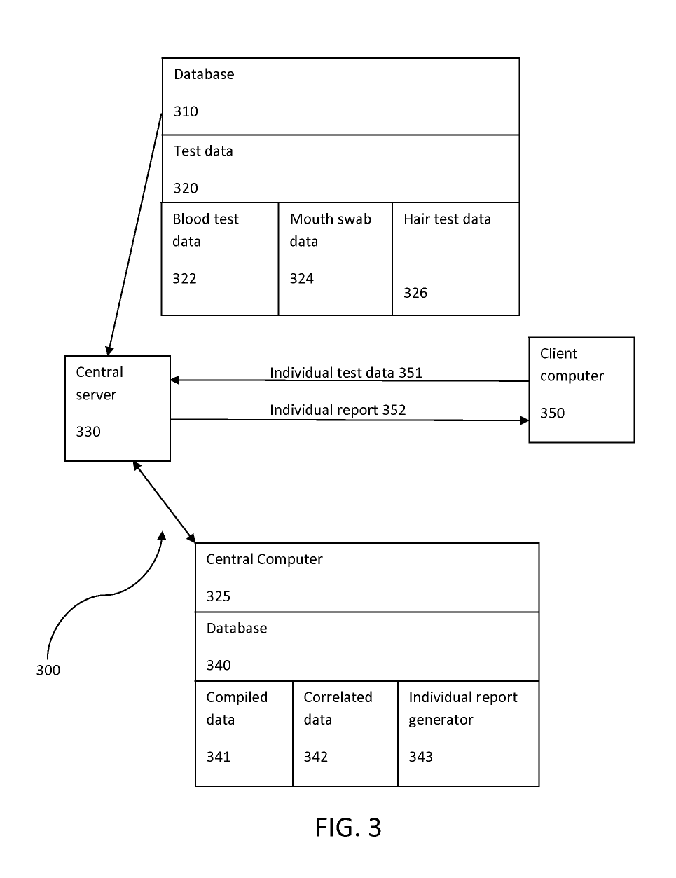

The usage of these methods may also be used for the design of devices to correlate the relationship between the various substances contained in the body as listed in this invention. These devices can include but should not be limited to ones used within the veins and arteries, ongoing assessment as implanted devices, monitoring devices for home use or those used to perform diagnostic assessments in physicians offices, hospitals, diagnostic laboratories, home care or any other setting where the analysis of these substances contained in the blood or other specimens used for analysis are obtained for measurement of the levels and then compared to each other to perform assessments to maintain proper health and well being. A schematic of the system diagram is shown in FIG. 3.

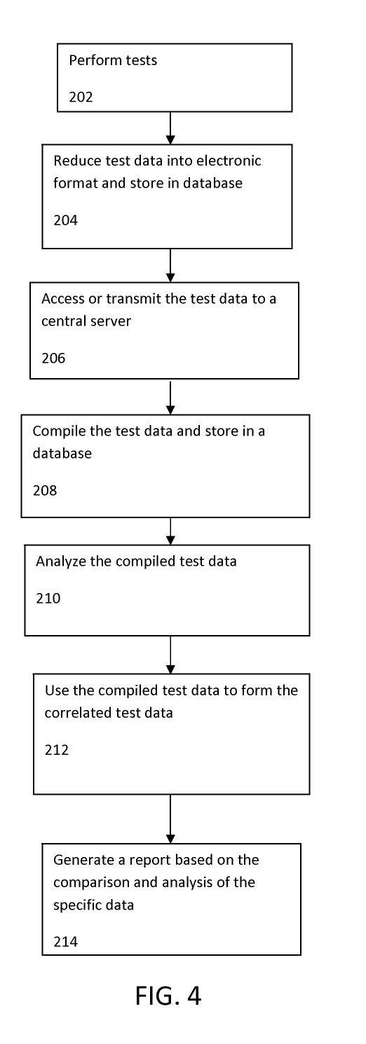

FIG. 3 shows a personalized report generator system 300 designed and arranged in accordance with an embodiment of the present invention. It should be understood that system 300 shows the basic components of the system and that more or less individualized components may be provided. FIG. 4 is a flow chart showing the steps involved in using the system 300.

The personalized report generator system 300 comprises a database 310 in which may be stored various test results. The data is stored in the database 310 as test data 320. The test data 320 is in a format that is capable of being manipulated by computers, processors, etc. The test data 320 may comprise various substance levels. In an exemplary embodiment of the present invention and shown in step 402, tests are performed on individual patients. In step 404, the results of these tests are then reduced into an electronic format in order to produce the test data 320. For example, the database 310 may store blood test data 322, mouth swab data 324 and hair test data 326. Other tests may include urine; fecal matter; collagen; chyle; interstitial fluid (tissue fluid); lymph; extracellular fluid; amniotic fluid; sweat tears; saliva; mucus; phlegm; hair; fingernails; bone marrow and breath. Each of these various tests may screen for a plurality of substances that are mentioned above in the list of modulated associated substances. As an example, the blood test data 322 may screen for iron 10, sulfur 12 and phosphorus 14.

The test data 320 may be organized and stored at the database 310. Alternatively, satellite locations may each maintain their own databases 310 that control their own records. In step 406, these locations may be accessed periodically by the central server 330 or the various databases 310 may transmit their results to a central location that is then accessible by a central server 330. Preferably the test data 320 is only accessed or transmitted when a certain threshold number of results are obtained. For example the test data 320 may not be sent to the central server 330 until at least one hundred tests are prepared to be transmitted.

In step 408, the transmitted test data 320 is compiled and stored as compiled test data 341. The central server 330 may keep the sent or accessed compiled test data 341 stored at its location in the central database 340, or the system 300 may provide a distributed storage system that stores the compiled test data 341 at external locations as well as the central database 340. However it should be understood that when compiled test data 341 is stored at distributed sites the system still functions as if they were stored at the central database 340. The compiled test data 341 may be stored so that the origin of the data is maintained in an anonymous fashion. In some instances tracking of the origin of a test result may only occur when a personalized report is requested.

In step 410, the compiled test data 341 is then analyzed in order to determine significant figures for conducting analysis. The analysis may be accomplished with the usage of a computer(s) 325 of which the server 330 may be a part of, as well as the database 310. The analysis may be accomplished by statistically compiling and correlating the results for one specific test or by compiling the data for two separate items that are believed to have a relationship. Specific values from the plurality of compiled test data 441 having a similar feature may be analyzed. This may be accomplished through the application of known statistical algorithms. In step 412, the statistically compiled test data 341 may be used to form the correlated data 342. In step 414, the statistically correlated data 342 is then used by the individual report generator 343 to form the personalized medical prescription and diagnosis. Upon receipt of individual test data 351 from a client computer 350, the individual report generator 343 processes the data and transmits an individual report 352 to a client computer 350.

The compiled test data 341 may be periodically updated from the databases 310 and through the addition of more collected test data. The updating of the compiled test data 341 may provide additional information with which to refine the correlated data 342 and to refine the ranges used by the individual report generator 343 when producing the personalized medical prescription and diagnosis. Additionally the updating of the compiled test data 341 may be a continuous feature wherein the addition of new information is immediately reflected in the correlated data 342. This may provide a constant analysis of compiled test data 341 that may reflect changes in population and diets in society.

It should be understood that all modulated associated substances disclosed above may be treated and analyzed in the same fashion as that discussed with iron 10, sulfur 12 and phosphorus 14 that comprise SIRT3.

Still yet another embodiment of the present invention may be a method for establishing a test to assess the existing levels of the constituents necessary to form the signaling molecule SIRT 1 in an individual comprising: measuring a level of phosphorus in the individual; measuring a level of lysine in the individual; measuring a level of sulfur in the individual; comparing the measured levels and means for establishing a treatment based upon the measured levels.

Still yet another embodiment of the present invention may be a method for establishing a test to assess the existing levels of the constituents necessary to form the signaling molecule SIRT 2 in an individual comprising: measuring a level of phosphorus in the individual; measuring a level of lysine in the individual; measuring a level of silicon in the individual; comparing the measured levels and means for establishing a treatment based upon the measured levels.

Still yet another embodiment of the present invention may be a method for establishing a test to assess the existing levels of the constituents necessary to form the signaling molecule SIRT 4 in an individual comprising: measuring a level of phosphorus in the individual; measuring a level of cysteine in the individual; measuring a level of sulfur in the individual; comparing the measured levels and means for establishing a treatment based upon the measured levels.

Still yet another embodiment of the present invention may be a method for establishing a test to assess the existing levels of the constituents necessary to form the signaling molecule SIRT 5 in an individual comprising: measuring a level of phosphorus in the individual; measuring a level of cysteine in the individual; measuring a level of silicon in the individual; comparing the measured levels and means for establishing a treatment based upon the measured levels.

Still yet another embodiment of the present invention may be a method for establishing a test to assess the existing levels of the constituents necessary to form the signaling molecule SIRT 6 in an individual comprising: measuring a level of phosphorous in the individual; measuring a level of cysteine in the individual; measuring a level of magnesium in the individual; comparing the measured levels; and establishing a treatment based upon the measured levels.

Still yet another embodiment of the present invention may be a method for establishing a establishing a test to assess the existing levels of the constituents necessary to form the signaling molecule SIRT 7 in an individual comprising: measuring a level of phosphorous in the individual; measuring a level of lysine in the individual; measuring a level of magnesium in the individual; comparing the measured levels; and establishing a treatment based upon the measured levels.

Still yet another embodiment of the present invention may be a method for establishing a test to assess the existing levels of the constituents that form one of the signaling molecules for cellular respiration in an individual comprising: measuring a level of sulfur in the individual; measuring a level of nickel in the individual; measuring a level of iron in the individual; comparing the measured levels; and establishing a treatment based upon the measured levels.

Still yet another embodiment of the present invention may be a method for establishing a test to assess the existing levels of the constituents that form another signaling molecule for cellular respiration in an individual comprising: measuring a level of sulfur in the individual; measuring a level of molybdenum in the individual; measuring a level of iron in the individual; comparing the measured levels; and establishing a treatment based upon the measured levels.

Still yet another embodiment of the present invention may be a method for establishing a test to assess the existing levels of the constituents that form a signaling molecule for blood oxidation in an individual comprising: measuring a level of sulfur in the individual; measuring a level of histidine in the individual; measuring a level of iron in the individual; comparing the measured levels; and establishing a treatment based upon the measured levels.

Still yet another embodiment of the present invention may be a method for establishing a test to assess the existing levels of the constituents that form a signaling molecule for iron metabolism in blood of an individual comprising: measuring a level of copper in the individual; measuring a level of zinc in the individual; measuring a level of iron in the individual; comparing the measured levels; and establishing a treatment based upon the measured levels.

Still yet another embodiment of the present invention may be a method for establishing a test to assess the existing levels of the constituents that form the signaling molecule eumelanin comprising: measuring a level of copper in the individual; measuring a level of serine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule phenomelanin comprising: measuring a level of copper in the individual; measuring a level of threonine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule neuromelanin comprising: measuring a level of copper in the individual; measuring a level of cysteine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule that is one of the forms of vitamin B6 comprising: measuring a level of copper in the individual; measuring a level of aspartic acid in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule that is another one of the forms of vitamin B6 comprising: measuring a level of copper in the individual; measuring a level of alanine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule that is another one of the forms of vitamin B6 comprising: measuring a level of copper in the individual; measuring a level of glutamic acid in the individual; measuring a level of zinc in the individual; comparing the measured levels; and establishing a treatment based upon the measured levels.

Still yet another embodiment of the present invention may be a method for establishing a test to assess the existing levels of cyanocobalamin in an individual comprising: measuring a level of cobalt in the individual; measuring a level of alanine in the individual; measuring a level of cyanide in the individual; comparing the measured levels; and establishing a treatment based upon the measured levels.

Still yet another embodiment of the present invention may be a method for establishing a test to assess the existing levels of proteins for cell adhesion in an individual comprising: measuring a level of DSC3 in the individual; measuring a level of DSC2 in the individual; measuring a level of DSG1 in the individual; comparing the measured levels; and establishing a treatment based upon the measured levels.

Still yet another embodiment of the present invention may be a method for establishing a test to assess the existing levels of proteins for cell adhesion in an individual comprising: measuring a level of DSC1 in the individual; measuring a level of DSC2 in the individual; measuring a level of DSC4 in the individual; comparing the measured levels; and establishing a treatment based upon the measured levels.

Still yet another embodiment of the present invention may be a method for establishing a establishing a test to assess the existing levels of the constituents that form the signaling molecule for cadmium chelation in an individual comprising: measuring a level of zinc in the individual; measuring a level of cysteine in the individual; measuring a level of manganese in the individual; comparing the measured levels; and establishing a treatment based upon the measured levels.

Still yet another embodiment of the present invention may be a method for establishing a establishing a test to assess the existing levels of the constituents that form the signaling molecule for connective tissue formation in an individual comprising: measuring a level of silicon in the individual; measuring a level of calcium in the individual; measuring a level of gold in the individual; comparing the measured levels; and establishing a treatment based upon the measured levels.

Still yet another embodiment of the present invention may be a method for establishing a test to assess the existing levels of the constituents that form the signaling molecule for connective tissue formation in an individual comprising: measuring a level of silicon in the individual; measuring a level of quartz in the individual; measuring a level of oxygen in the individual; comparing the measured levels; and establishing a treatment based upon the measured levels.

Still yet another embodiment of the present invention may be a method for establishing a test to assess the existing levels of the constituents that form the signaling molecule dopamine in an individual: measuring a level of nickel in the individual comprising; measuring a level of phenylalanine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule norepinephrine comprising; measuring a level of nickel; measuring a level of tyrosine; measuring a level of zinc; and establishing a treatment based upon the measured levels.

Still yet another embodiment of the present invention may be a method for establishing a test to assess the existing levels of the constituents that form the signaling molecule adrenaline comprising; measuring a level of nickel; measuring a level of tryptophan; measuring a level of zinc; and establishing a treatment based upon the measured levels.

Still yet another embodiment of the present invention may be a method for establishing a test to assess the existing levels of signaling molecules for connective tissue in an individual comprising: measuring a level of thrombospondin 1 in the individual; measuring a level of thrombospondin 2 in the individual; measuring a level of thrombospondin 3 in the individual; comparing the measured levels; and establishing a treatment based upon the measured levels.

Still yet another embodiment of the present invention may be a method for establishing a test to assess the existing levels of signaling molecules for connective tissue in an individual comprising: measuring a level of thrombospondin 4 in the individual; measuring a level of thrombospondin 2 in the individual; measuring a level of thrombospondin 5 in the individual; comparing the measured levels; and establishing a treatment based upon the measured levels.

Still yet another embodiment of the present invention may be a method for establishing a test to assess the existing levels of proteins for synapse formation in an individual comprising: measuring a level of NLGN3 in the individual; measuring a level of NLGN2 in the individual; measuring a level of NLGN5 in the individual; comparing the measured levels; and establishing a treatment based upon the measured levels.

Still yet another embodiment of the present invention may be a method for establishing a test to assess the existing levels of proteins for synapse formation in an individual comprising: measuring a level of NLGN1 in the individual; measuring a level of NLGN2 in the individual; measuring a level of NLGN4X in the individual; comparing the measured levels; and establishing a treatment based upon the measured levels.

Still yet another embodiment of the present invention may be a method for establishing a test to assess the existing levels of signaling molecules for synapse adhesion in an individual comprising: measuring a level of NRXN2 in the individual; measuring a level of NRXN1 in the individual; measuring a level of NGF in the individual; comparing the measured levels; and establishing a treatment based upon the measured levels.

Still yet another embodiment of the present invention may be a method for establishing a test to assess the existing levels of signaling molecules for synapse adhesion in an individual comprising: measuring a level of NRXN3 in the individual; measuring a level of NRXN1 in the individual; measuring a level of NRXN4 in the individual; comparing the measured levels; and establishing a treatment based upon the measured levels.

Still yet another embodiment of the present invention may be a method for establishing a test to assess the existing levels of the constituents that form the signaling molecule vitamin K1 in an individual comprising: measuring a level of phosphorous in the individual; measuring a level of isoleucine in the individual; measuring a level of calcium in the individual; comparing the measured levels; and establishing a treatment based upon the measured levels.

Still yet another embodiment of the present invention may be a method for establishing a test to assess the existing levels of the constituents that form the signaling molecule vitamin K2 in an individual comprising: measuring a level of phosphorous in the individual; measuring a level of leucine in the individual; measuring a level of calcium in the individual; comparing the measured levels; and establishing a treatment based upon the measured levels.

Still yet another embodiment of the present invention may be a method for establishing a test to assess the existing levels of the constituents that form the signaling molecule MK4 form of vitamin K in an individual comprising: measuring a level of phosphorous in the individual; measuring a level of valine in the individual; measuring a level of calcium in the individual; comparing the measured levels; and establishing a treatment based upon the measured levels.

Still yet another embodiment of the present invention may be a method for establishing a test to assess the existing levels of the constituents that form one of the forms of the signaling molecule vitamin B1 in an individual comprising: measuring a level of sulfur in the individual; measuring a level of phenylalanine in the individual; measuring a level of iron in the individual; comparing the measured levels; and establishing a treatment based upon the measured levels.

Still yet another embodiment of the present invention may be a method for establishing a test to assess the existing levels of the constituents that form another of the forms of the signaling molecule vitamin B1 in an individual comprising: measuring a level of sulfur in the individual; measuring a level of tyrosine in the individual; measuring a level of iron in the individual; comparing the measured levels; and establishing a treatment based upon the measured levels.

Still yet another embodiment of the present invention may be a method for establishing a test to assess the existing levels of the constituents that form another of the forms of the signaling molecule vitamin B1 in an individual comprising: measuring a level of sulfur in the individual; measuring a level of tryptophan in the individual; measuring a level of iron in the individual; comparing the measured levels; and establishing a treatment based upon the measured levels.

Still yet another embodiment of the present invention may be a method for establishing a test to assess the existing levels of the constituents that form one of the forms of the signaling molecule vitamin B2 in an individual comprising: measuring a level of sulfur in the individual; measuring a level of leucine in the individual; measuring a level of iron in the individual; comparing the measured levels; and establishing a treatment based upon the measured levels.

Still yet another embodiment of the present invention may be a method for establishing a test to assess the existing levels of the constituents that form another of the forms of the signaling molecule vitamin B2 in an individual comprising: measuring a level of sulfur in the individual; measuring a level of isoleucine in the individual; measuring a level of iron in the individual; comparing the measured levels; and establishing a treatment based upon the measured levels.

Still yet another embodiment of the present invention may be a method for establishing a test to assess the existing levels of the constituents that form another one of the forms of the signaling molecule vitamin B2 in an individual comprising: measuring a level of sulfur in the individual; measuring a level of valine in the individual; measuring a level of iron in the individual; comparing the measured levels; and establishing a treatment based upon the measured levels.

Still yet another embodiment of the present invention may be a method for establishing a test to assess the existing levels of the constituents that form one of the forms of the signaling molecule vitamin B3 in an individual comprising: measuring a level of iron in the individual; measuring a level of phenylalanine in the individual; measuring a level of manganese in the individual; comparing the measured levels; and establishing a treatment based upon the measured levels.

Still yet another embodiment of the present invention may be a method for establishing a test to assess the existing levels of the constituents that form another of the forms of the signaling molecule vitamin B3 in an individual comprising: measuring a level of iron in the individual; measuring a level of tyrosine in the individual; measuring a level of manganese in the individual; comparing the measured levels; and establishing a treatment based upon the measured levels.

Still yet another embodiment of the present invention may be a method for establishing a test to assess the existing levels of the constituents that form another one of the forms of the signaling molecule vitamin B3 in an individual comprising: measuring a level of iron in the individual; measuring a level of tryptophan in the individual; measuring a level of manganese in the individual; comparing the measured levels; and establishing a treatment based upon the measured levels.

Still yet another embodiment of the present invention may be a method for establishing a test to assess the existing levels of the constituents that form one of the forms of the signaling molecule vitamin B5 in an individual comprising: measuring a level of copper in the individual; measuring a level of histidine in the individual; measuring a level of zinc in the individual; comparing the measured levels; and establishing a treatment based upon the measured levels.

Still yet another embodiment of the present invention may be a method for establishing a test to assess the existing levels of the constituents that form another of the forms of the signaling molecule vitamin B5 in an individual comprising: measuring a level of copper in the individual; measuring a level of arginine in the individual; measuring a level of zinc in the individual; comparing the measured levels; and establishing a treatment based upon the measured levels.

Still yet another embodiment of the present invention may be a method for establishing a test to assess the existing levels of the constituents that form another of the forms of the signaling molecule vitamin B5 in an individual comprising: measuring a level of copper in the individual; measuring a level of lysine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule vitamin B12 in an individual comprising: measuring a level of manganese in the individual; measuring a level of cyanocobalamin in the individual; measuring a level of iron in the individual; comparing the measured levels; and establishing a treatment based upon the measured levels.

Still yet another embodiment of the present invention may be a method for establishing a test to assess the existing levels of the constituents that form the signaling molecule melatonin in an individual comprising: measuring a level of tryptophan in the individual; measuring a level of nitric oxide in the individual; measuring a level of phosphorus in the individual; comparing the measured levels; and establishing a treatment based upon the measured levels.

Still yet another embodiment of the present invention may be a method for establishing a test to assess the existing levels of the constituents that form the signaling molecule choline in an individual comprising; measuring a level of phosphorous in the individual; measuring a level of lysine in the individual; measuring a level of calcium in the individual; comparing the measured levels; and establishing a treatment based upon the measured levels.

Still yet another embodiment of the present invention may be a method for establishing a test to assess the existing levels of the constituents that form one of the forms of the signaling molecule glutathione peroxidase in an individual comprising: measuring a level of selenium in the individual; measuring a level of threonine in the individual; measuring a level of zinc in the individual; comparing the measured levels; and establishing a treatment based upon the measured levels.

Still yet another embodiment of the present invention may be a method for establishing a test to assess the existing levels of the constituents that form another one of the forms of the signaling molecule glutathione peroxidase in an individual comprising: measuring a level of selenium in the individual; measuring a level of serine in the individual; measuring a level of zinc in the individual; comparing the measured levels; and establishing a treatment based upon the measured levels.

Still yet another embodiment of the present invention may be a method for establishing a test to assess the existing levels of the constituents that form another one of the forms of the signaling molecule glutathione peroxidase in an individual comprising: measuring a level of selenium in the individual; measuring a level of cysteine in the individual; measuring a level of zinc in the individual; comparing the measured levels; and establishing a treatment based upon the measured levels.

Still yet another embodiment of the present invention may be a method for establishing a test to assess the existing levels of the constituents that form one of the forms of the signaling molecule vitamin B7 in an individual comprising: measuring a level of copper in the individual; measuring a level of leucine in the individual; measuring a level of zinc in the individual; comparing the measured levels; and establishing a treatment based upon the measured levels.

Still yet another embodiment of the present invention may be a method for establishing a test to assess the existing levels of the constituents that form another one of the forms of the signaling molecule vitamin B7 in an individual comprising: measuring a level of copper in the individual; measuring a level of isoleucine in the individual; measuring a level of zinc in the individual; comparing the measured levels; and establishing a treatment based upon the measured levels.

Still yet another embodiment of the present invention may be a method for establishing a test to assess the existing levels of the constituents that form another one of the forms of the signaling molecule vitamin B7 in an individual comprising: measuring a level of copper in the individual; measuring a level of valine in the individual; measuring a level of zinc in the individual; comparing the measured levels; and establishing a treatment based upon the measured levels.

Still yet another embodiment of the present invention may be a method for establishing a test to assess the existing levels of proteins for cell proliferation and adhesion in an individual comprising: measuring a level of an EGFR2; measuring a level of an EGFR1; measuring a level of GRB7; and establishing a treatment based upon the measured levels.

Still yet another embodiment of the present invention may be a method for establishing a test to assess the existing levels of proteins for cell proliferation in an individual comprising: measuring a level of EGFR3; measuring a level of EGFR1; measuring a level of EGFR4; and establishing a treatment based upon the measured levels.

Still yet another embodiment of the present invention may be a method for establishing a test to assess the existing levels of the constituents that form the signaling molecule Estradiol (E2) comprising; measuring a level of selenium; measuring a level of phenylalanine; measuring a level of zinc; and establishing a treatment based upon the measured levels.

Still yet another embodiment of the present invention may be a method for establishing a test to assess the existing levels of the constituents that form the signaling molecule Estrone (E1) comprising; measuring a level of selenium; measuring a level of tyrosine; measuring a level of zinc; and establishing a treatment based upon the measured levels.

Still yet another embodiment of the present invention may be a method for establishing a test to assess the existing levels of the constituents that form the signaling molecule Estriol (E3) comprising; measuring a level of selenium; measuring a level of tryptophan; measuring a level of zinc; and establishing a treatment based upon the measured levels.

Still yet another embodiment of the present invention may be a method for establishing a test to assess the existing levels of the constituents that form the signaling molecule serotonin comprising; measuring a level of sulfur; measuring a level of histidine; measuring a level of iron; and establishing a treatment based upon the measured levels.

Still yet another embodiment of the present invention may be a method for establishing a test to assess the existing levels of the constituents that form the signaling molecule oxytocin comprising; measuring a level of sulfur; measuring a level of arginine; measuring a level of iron; and establishing a treatment based upon the measured levels.

Still yet another embodiment of the present invention may be a method for establishing a test to assess the existing levels of the constituents that form the signaling molecule prolactin comprising; measuring a level of sulfur; measuring a level of lysine; measuring a level of iron; and establishing a treatment based upon the measured levels.

Still yet another embodiment of the present invention may be a method for establishing a test to assess the existing levels of the constituents that form the signaling molecule cortisol comprising; measuring a level of zinc; measuring a level of nitric oxide; measuring a level of nickel; and establishing a treatment based upon the measured levels.

Still yet another embodiment of the present invention may be a method for establishing a test to assess the existing levels of the constituents that form the Ergosterol form of signaling molecule vitamin D comprising: measuring a level of phosphorus in the individual; measuring a level of threonine in the individual; measuring a level of calcium in the individual; comparing the measured levels; and establishing a treatment based upon the measured levels.

Still yet another embodiment of the present invention may be a method for establishing a test to assess the existing levels of the constituents that form signaling molecule vitamin D2 comprising: measuring a level of phosphorus in the individual; measuring a level of serine in the individual; measuring a level of calcium in the individual; comparing the measured levels; and establishing a treatment based upon the measured levels.

Still yet another embodiment of the present invention may be a method for establishing a test to assess the existing levels of the constituents that form signaling molecule vitamin D3 comprising: measuring a level of phosphorus in the individual; measuring a level of cysteine in the individual; measuring a level of calcium in the individual; comparing the measured levels; and establishing a treatment based upon the measured levels.

Still yet another embodiment of the present invention may be a method for establishing a test to assess the existing levels of the constituents that form one of the forms of signaling molecule vitamin B9 comprising: measuring a level of phosphorus in the individual; measuring a level of serine in the individual; measuring a level of sulfur in the individual; comparing the measured levels; and establishing a treatment based upon the measured levels.

Still yet another embodiment of the present invention may be a method for establishing a test to assess the existing levels of the constituents that form another one of the forms of signaling molecule vitamin B9 comprising: measuring a level of phosphorus in the individual; measuring a level of threonine in the individual; measuring a level of sulfur in the individual; comparing the measured levels; and establishing a treatment based upon the measured levels.

Still yet another embodiment of the present invention may be a method for establishing a test to assess the existing levels of the constituents that form the signaling molecule Fas comprising: measuring a level of calcium in the individual; measuring a level of histidine in the individual; measuring a level of magnesium in the individual; comparing the measured levels; and establishing a treatment based upon the measured levels.

Still yet another embodiment of the present invention may be a method for establishing a test to assess the existing levels of the constituents that form the signaling molecule Ras comprising: measuring a level of calcium in the individual; measuring a level of arginine in the individual; measuring a level of magnesium in the individual; comparing the measured levels; and establishing a treatment based upon the measured levels.

Still yet another embodiment of the present invention may be a method for establishing a test to assess the existing levels of the constituents that form the signaling molecule Bax comprising: measuring a level of calcium in the individual; measuring a level of lysine in the individual; measuring a level of magnesium in the individual; comparing the measured levels; and establishing a treatment based upon the measured levels.

Still yet another embodiment of the present invention may be a method for establishing a test to assess the existing levels of the constituents that form the signaling molecule Ax1 comprising: measuring a level of nickel in the individual; measuring a level of phenylalanine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule Tyro3 comprising: measuring a level of nickel in the individual; measuring a level of tyrosine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule Mer comprising: measuring a level of nickel in the individual; measuring a level of tryptophan in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule Histone H3.1 comprising: measuring a level of nickel in the individual; measuring a level of histidine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule Histone H3.2 comprising: measuring a level of nickel in the individual; measuring a level of arginine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule Histone H3.3 comprising: measuring a level of nickel in the individual; measuring a level of lysine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule Pregnenolone (PREG) comprising: measuring a level of calcium in the individual; measuring a level of glutamic acid in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule Dehydroepiandrosterone (DHEA) comprising: measuring a level of calcium in the individual; measuring a level of alanine in the individual; measuring a level of zinc in the individual; comparing the measured levels; and establishing a treatment based upon the measured levels.

Still yet another embodiment of the present invention may be a method for establishing a test to assess the existing levels of the constituents that form the signaling molecule Progesterone (PROG) comprising: measuring a level of calcium in the individual; measuring a level of aspartic acid in the individual; measuring a level of zinc in the individual; comparing the measured levels; and establishing a treatment based upon the measured levels.

Still yet another embodiment of the present invention may be a method for establishing a test to assess the existing levels of proteins for the formation of factors in cell proliferation and differentiation comprising: measuring a level of transforming growth factor b-2 (TGFb-2); measuring a level of transforming growth factor b-3 (TGFb-3); measuring a level of p53; and establishing a treatment based upon the measured levels.

Still yet another embodiment of the present invention may be a method for establishing a test to assess the existing levels of the constituents that form the signaling molecule Interleukin 4 comprising: measuring a level of nickel; measuring a level of zinc; measuring a level of boron; and establishing a treatment based upon the measured levels.

Still yet another embodiment of the present invention may be a method for establishing a test to assess the existing levels of the constituents that form the signaling molecule Interleukin 6 comprising: measuring a level of calcium; measuring a level of magnesium; measuring a level of chloride; and establishing a treatment based upon the measured levels.

Still yet another embodiment of the present invention may be a method for establishing a test to assess the existing levels of the constituents that form the signaling molecule Interleukin 10 comprising: measuring a level of bromine; measuring a level of iodine; measuring a level of chloride; and establishing a treatment based upon the measured levels.

Still yet another embodiment of the present invention may be a method for establishing a test to assess the existing levels of proteins for the formation of factors in cell proliferation and differentiation comprising: measuring a level of transforming growth factor b-4 (TGFb-4); measuring a level of transforming growth factor b-3 (TGFb-3); measuring a level of transforming growth factor b-1 (TGFb-1); and establishing a treatment based upon the measured levels.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A method of for providing homeostasis of SIRT 3 via the levels of substances that form SIRT 3 in an individual comprising:
measuring a level of iron in the individual;
measuring a level of sulfur in the individual;
measuring a level of phosphorus in the individual;
comparing the measured levels; and
establishing homeostasis of SIRT 3 based upon the measured levels.

2. The method of claim 1, further comprising administering iron to the individual.

3. The method of claim 1, further comprising reducing intake of iron.

4. The method of claim 1, wherein the measuring is accomplished with a blood test.

5. The method of claim 1, wherein test data is stored and compiled in a central computer.

6. The method of claim 1, wherein the levels of iron, phosphorous and sulfur are re-measured.

7. A method of for providing homeostasis of SIRT 3 via the levels of substances that form SIRT 3 in an individual comprising:
means for measuring a level of phosphorous in the individual;
means for measuring a level of iron in the individual;
means for comparing the measured levels; and
means for establishing homeostasis of SIRT 3 based upon the measured levels.

8. The system of claim 7, further comprising administering iron to the individual.

9. The system of claim 7, further comprising means for reducing the intake of iron.

10. The system of claim 7, wherein the means for measuring is accomplished with a blood test.

11. The system of claim 7, wherein test data is stored and compiled in a central computer.

12. The system of claim 7, wherein the levels of iron, phosphorous and sulfur are re-measured.
